(19) United States
(12) Patent Application Publication
(10) Pub. No.: US 2011/0129906 A1
(43) Pub. Date: Jun. 2, 2011

(54) PHOTOBIOREACTOR, SYSTEM AND METHOD FOR THE CULTIVATION OF PHOTOSYNTHETIC MICROORGANISMS

(76) Inventor: Nahshon Edelson, D.N. Hanegev (IL)

(21) Appl. No.: 12/997,990

(22) PCT Filed: Jun. 18, 2009
(86) PCT No.: PCT/IL2009/000606

§ 371 Date: Dec. 14, 2010

(30) Foreign Application Priority Data
Jun. 19, 2008 (IL) 192333

(51) Int. Cl. C12N 1/12; C12M 1/00

(52) U.S. Cl. 435/257.1; 435/292.1

(57) ABSTRACT

The present invention relates to the cultivation of photosynthetic microorganisms, and more particularly, to a low-cost flexible photobioreactor (1), as well as systems and methods using the same for optimizing the growth of microalgal species. The photobioreactor of the invention is in the shape of a flexible transparent elongated body (6) adapted to be positioned horizontally in a body of water, and comprises i) an elongated gas dispensing system (7) for providing nutrients by means of bubbling a gas mixture to a liquid suspension and for performing a vertical low stress mixing of said liquid suspension, ii) an elongated filling/draining system (11) for controlling the volume of said liquid suspension in the photobioreactor, and iii) at least one gas evacuation opening (13).

### FIELD OF THE INVENTION

The present invention relates to the cultivation of photosynthetic microorganisms, and more particularly, to a low-cost flexible photobioreactor, a system and a method thereof for optimizing the growth of microalgal species.

### BACKGROUND OF THE INVENTION

Microalgal biotechnology only started in the middle of the last century but has grown and diversified significantly in the last thirty years. Commercial large-scale culture begun in the early 1960's in Japan with the culture of Chlorella by Nihon Chlorella. Nowadays, the microalgal biomass market produces about 5000 t of dry matter/year and generates a turnover of approximately US$ 1.25×10⁹ /year (Spolaore et al., Journal of Bioscience and Bioengineering, Vol. 101(2), pp. 87-96, 2006). The special chemical composition of microalgae makes them very attractive for the food industry, aquaculture, cosmetics, and biofuel.

Microalgae are able to synthesize all the amino acids and may provide the essential ones to humans and animals. Carbohydrates are available in the form of starch, glucose or other types of polysaccharides, and represent 10% to 60% of the total dry weight. The average lipid content, comprising glycerol and sugars or bases esterified with saturated or unsaturated fatty acids, varies between 1% and 70%. Among all the fatty acids, some belong to the ω3 and ω6 families, which are of particular interest. Microalgae also represent a valuable source of almost all vitamins (e.g., A, B1, B2, B6, B12, C, E, nicotinate, biotin, folic acid and pantothenic acid). Vitamins improve the nutritional value of algal cells, but their quantity fluctuates with environmental factors, with the harvest treatment and with the drying method. Microalgae are also rich in pigments like chlorophyll (0.5% to 1% of dry weight), carotenoids (0.1% to 0.2% of dry weight on average) and phycobiliproteins.

Microalgae are of particular interest in the field of “green” energy as they can provide several type of renewable biofuels. These include methane produced by anaerobic digestion of the algal biomass; biodiesel derived from the algal oil; and hydrogen produced photobiologically. However, replacing the transport fuel would require at least a half of billion m³ of biodiesel annually in the US alone, at current consumption rates (Yussuf Chisti, Biodiesel from microalgae, Biotech. Adv., Vol. 25, pp. 294-306, 2007). Biodiesel, which is currently produced from higher plants oil (corn, soybean, etc.) and animal fat, can not realistically match this demand as it would require large cultivation areas and high production costs. Unlike the other oil crops, microalgae can be grown rapidly, require a smaller space to grow, many are extremely rich in oil, and their production may potentially make use of gas exhausted from power plant (CO₂, NO₂, etc.).

Nowadays, successful large-scale commercial production of microalgal biomass is done in open ponds. Building costs are low (few $ per m²) but this cultivation system presents several disadvantages. It is currently limited to a few microalgal species as most microalgae cannot be maintained outdoor due to the risks of contamination. Furthermore, light penetration and dispersion in open ponds is not optimal as it creates an exposure gradient in the culture medium, the amount of available light received decreasing with the depth of the pond. Open ponds can not maintain or regulate the growth temperature, and mixing of the growth solution is only achieved by linear flow. Therefore, photobioreactors (PBR), which offer a closed and monitored culture environment, are currently developed in order to optimize the production efficiency and to enable the potential exploitation of more microalgal species.

The key factors when designing a PBR are: surface-to-volume ratio, orientation, inclination, mixing and degassing devices, cleaning systems, temperature regulation, transparency and durability of the container. The ease of operation, scale-up, low construction and operating costs are also particularly relevant when directed to commercial PBR (Tredici M., Handbook of Microalgal Culture: Biotechnology and Applied Phycology, chapter 9, Blackwell Publishing Ltd., 2004). Achieving a good mixing of the growth solution is particularly important as it prevents biofouling and thermal stratification, breaking down the diffusion gradient at the cell surface, helping to decrease the concentration of dissolved oxygen generated during photosynthesis, easing the distribution of nutrients, and ensuring that cells experience alternating periods of light and darkness without high shearing stress.

In term of design, the main categories of reactors are: flat or tubular; horizontal, inclined, vertical or spiral, manifold or serpentine. An operational classification of PBR would include air vs. pump mixing, and single-phase reactor (filled with media, with gas exchange taking place in a separate gas exchanger) vs. two-phase reactors (in which both gas and liquid are present and continuous gas mass transfer takes place in the reactor itself). Construction materials provide additional variation and subcategories, for example, glass vs. plastic, and rigid vs. flexible. Nowadays, the installation cost of commercial PBRs for large scale algal biomass production remains dissuasive (several 100$ per m² in average), and asks for consequent investments without guarantee of success. A general view of existing systems can be approached by reviewing the following publications.

GB 2117572 relates to an horizontal tubular photobioreactor, of which design served as a model for the implementation of a commercial scale PBR in Spain (Photo Bioreactors Ltd.), using 1.2 cm diameter, 50 m long rigid polyethylene tubes connected to vertical manifolds. The circulation is made by airlift and the temperature control via shading the tubes with nets or water spraying. However, the small diameter of the tubes, avoiding effective mixing, the very high s/v ratio and an inefficient degassing system of oxygen produced by the culture resulted in poor algal growth, biofouling, and heavy contamination. Furthermore, the temperature regulating system proved to be inefficient since shading, to be effective, requires that a large portion of the reactor (up to 80%) be covered during the hours of maximum insolation, which causes a significant reduction of productivity.

U.S. Pat. No. 3,955,317 relates to a horizontal tubular serpentine photobioreactor based on low density polyethylene connected tubes, supported by a body of water. Thermal control is achieved by regulating the buoyancy of the system by introducing water or air in floating means attached under the culture containers or to a rafting structure. However, the maintenance of oxygen levels below the toxic concentration requires frequent degassing in serpentines PBR and thus requires very short loops or high flow rates, making this design power consuming and difficult to scale up.

U.S. Pat. No. 4,868,123 relates to a horizontal tubular manifold photobioreactor based on polyethylene tubes, aligned in parallel and placed on an expanse of water. A second set of tubes is located beneath the first one by Y-shaped means, controlling the buoyancy of the system by inflation/deflation. Carbon dioxide is injected in the medium by a carbonator connected to the PBR inlet, and oxygen resulting from photosynthesis is removed by a complex degassing system connected to each single tube. Mixing of the microalgal culture is realized only by the flow generated by introducing the medium into the PBR. The overall system is complex and costly to implement in large scale. Furthermore, no specific attention is paid to the mixing which is, as shown previously, a key factor in the effective cultivation of microalgae.

U.S. Pat. No. 5,534,417 relates to disposable vertical photobioreactors, which are made of polyethylene sleeves hung on an solid structure and wherein mixing is achieved by bubbling air from the bottom. The main drawback of this culture system is the need of a heavy and costly structure used to support almost 250 kg/m² of growth solution, and the complex tubular systems for providing CO₂ and, air for collecting the growth medium. Furthermore, this system, when used outdoor, is presenting a large angle to the sun's rays, for which a substantial amount of solar energy is reflected and not available for growth.

The inventors believe that the present invention can solve many of the problems above-discussed in the prior art. Therefore it is an object to this invention to provide a low cost photobioreactor for the efficient growth of microalgae or other photosynthetic microorganisms.

It is another object to this invention to provide a low-cost system for the large-scale production of microalgal biomass.

It is still another object to the present invention to provide a method for the cultivation of microalgal biomass in industrial amounts.

It is still another object to the present invention to provide a method for the collection of the microalgal biomass from the photobioreactor of the invention.

Other objects and advantages of present invention will appear as description proceeds.

### SUMMARY OF THE INVENTION

In a first aspect, the invention relates to an horizontal sleeve photobioreactor (PBR) for growing photosynthetic microorganisms, comprising: i) a flexible transparent elongated body suitable to contain a liquid suspension of photosynthetic microorganisms, and adapted to be positioned horizontally in a body of water; ii) an elongated gas dispensing system for providing nutrients by means of bubbling a gas mixture to a liquid suspension and for achieving a vertical low stress mixing of the liquid suspension all along the elongated body; iii) an elongated filling/draining system for providing a growth medium and collecting the liquid suspension all along the elongated body, and iv) at least one gas evacuation opening. The photobioreactor of the invention is characterized in that both the cross section and the buoyancy of the flexible elongated transparent body can be modified in real-time by adjusting the volume of gas and liquid contained inside.

In a particular embodiment of the PBR of the invention, the elongated gas dispensing system comprises a single gas dispensing tube which is placed at the bottom of said photobioreactor, and which may comprise one or more weights. In this embodiment, the gas dispensing tube comprises one or more gas apertures facing the bottom of the photobioreactor to avoid the solution entering in the gas dispensing system.

In another embodiment of the PBR of the invention, the elongated gas dispensing system comprises a first and a second gas dispensing tube, both of said tubes being placed at the bottom of said photobioreactor and comprising one or more gas apertures facing the bottom of said photobioreactor. In this embodiment, the first gas dispensing tube is suitable to provide large bubbles for achieving a low-stress mixing of said liquid suspension, and the second gas dispensing tube is suitable to provide microbubbles containing nutrients to said liquid suspension.

In still other embodiments of the PBR of the invention, the elongated gas dispensing system is placed in the upper part of the photobioreactor and comprises lateral microtubes. Each of the lateral microtubes comprises one or more gas apertures and is maintained vertically dipped into the growth solution, by either the addition of one or more weights, or by fixing them vertically to the draining tube situated at the bottom of the PBR, or to the PBR body itself.

In a particular embodiment of the PBR of the invention, the filling/draining system comprises a single tube with one or more apertures, which is used for both draining and filling said photobioreactor.

In other embodiments of the PBR of the invention, the filling/draining system comprises at least a draining tube and a filling tube, each tube comprising one or more apertures. The draining tube and the filling tube may be both placed at the bottom of said photobioreactor. In a specific embodiment, another configuration, the draining tube is placed at the bottom of said photobioreactor and the filling tube is placed in the upper part of the photobioreactor.

In the PBR of the invention, the gas mixture can be accumulated above the liquid suspension, thereby enabling changes in the shape configuration of said photobioreactor and changes of the buoyancy of said photobioreactor in said body of water.

In a particular embodiment, the PBR of the invention comprises the following elements:
i) an elongated containing body made of a transparent sleeve of a flexible material, containing photosynthetic microorganisms in a liquid suspension;
ii) an elongated gas dispensing system comprising at least one gas dispensing tube with one or more gas apertures;
iii) an elongated draining/filling system comprising at least one tube with one or more apertures; and
iv) at least one gas evacuation opening.
   The elongated containing body is closed at both ends and lays horizontally in a body of water, its cross-section and buoyancy being adapted according to the external conditions and the growth cycle of the microorganisms contained in it. The elongated containing body is made of a weldable material, such as polyethylene.

In some embodiments, the PBR of the invention comprises at least one floating means, which may be inflated or deflated to maintain the same level of buoyancy all along said transparent containing body in said body of water.

In some other embodiments, the PBR of the invention comprises an anchoring means which connects it to the bottom of an artificial water pond. In some particular embodiments, the anchoring means, the water ponds and the transparent elongated body of the photobioreactor are made of a single transparent flexible element.

In a further aspect, the present invention relates to a system for the large-scale production of microalgal biomass, the system comprising:
i) a plurality of photobioreactors as above-described;
ii) a miscellaneous gas providing system;
iii) an air providing system;
iv) a growth medium supplying system;
v) a storage system;
vi) a medium recycling system;
vii) a collecting system;
viii) a sanitizing system; and
ix) optionally, a degassing system.
   In the above system, the photobioreactors are laid horizontally in a body of water, and all the elements are connected by fluid conveying tubes.

In still a further aspect, the present invention relates to a method for the cultivation of a microalgal biomass, comprising growing microalgae in a photobioreactor as described above, wherein said photobioreactor is laying horizontally in a body of water, and adapting the amount of light delivered to said biomass as well as the growth temperature of said biomass by changing the shape configuration and/or the buoyancy of said photobioreactor in said body of water. More particularly, the method of cultivation of the present invention comprises the following steps:
i) introducing a liquid suspension of microalgae containing a growth medium into an elongated transparent photobioreactor laying horizontally in a body of water;
ii) exposing said bioreactor to light, allowing sufficient light to pass through said liquid suspension and enabling photosynthetic algae to perform photosynthesis;
iii) blowing into said solution a gas mixture containing carbon dioxide by means of a gas dispensing system with several gas apertures, providing nutrients for the photosynthetic process and mixing said solution by means of bubbles;
iv) filling or draining culture medium homogeneously along said photobioreactor in order to maintain a constant density of algal population; and
v) changing the shape configuration and the buoyancy of said photobioreactor in said water body according to the external conditions.

Preferably, the gas mixture is provided in pulse mode. Changes in the shape configuration and buoyancy of the photobioreactor allow to monitor the amount of light delivered to the algal biomass and the temperature of the liquid suspension. Changes in the shape configuration and the buoyancy of the photobioreactor can be performed by modifying the volume of gas contained above the liquid suspension, by modifying the volume of gas in floating means, by modifying the level of water, by changing the volume of growth solution, or any combination thereof.

In still a further aspect, the invention provides several methods for collecting the algal biomass from a photobioreactor as described above. A first method comprises the following steps:
i) closing the gas dispensing system as well as the gas evacuation openings;
ii) enabling the microalgae to flocculate and fall down to the bottom of said photobioreactor;
iii) draining out the solution containing said algal biomass around the draining tube;
iv) reopening the gas dispensing system as well as the gas evacuation openings;
v) introducing a fresh microalgal suspension in the remaining growth solution; and
vi) optionally, introducing a volume of enriched growth medium into said photobioreactor.

The second collection method comprises the following steps:
i) closing the gas dispensing system as well as the gas evacuation openings;
ii) enabling the microalgae to flocculate and fall down to the bottom of said photobioreactor;
iii) draining out the solution containing said algal biomass around the draining tube;
iv) draining out the remaining growth solution;
v) introducing a fresh microalgal suspension mixed to a fresh growth solution; and
vi) reopening the gas dispensing system as well as the gas evacuation openings.

A third collecting method comprises the following steps:
i) draining the whole growth solution from a first photobioreactor and dividing said solution in several volumes, each of said volume being used for filling several photobioreactors with an internal volume smaller than said first photobioreactor;
ii) collecting the algal biomass that flocculates near the draining tube in each of said smaller photobioreactors;
iii) collecting the remaining growth medium in each of said smaller photobioreactors; and
iv) introducing a fresh microalgal suspension mixed to a fresh growth solution into said first photobioreactor.
   In this method, filing and collecting of the solution is made according to a mode selected from the group consisting of parallel mode, direct mode and hybrid mode.

### BRIEF DESCRIPTION OF THE DRAWINGS

The above and other characteristics and advantages of the invention will be more readily apparent through the following examples, and with reference to the appended drawings, wherein:

FIGS. 1A and 1B schematically show a perspective view and a cross section view of one embodiment of the photobioreactor (PBR) of the invention, having a gas dispensing system placed a the bottom of said PBR;

FIGS. 2A and 2B schematically shows a perspective view and a cross section view of another embodiment of the photobioreactor of the invention, having a gas dispensing system placed a the top of said PBR;

FIGS. 3A to 3C schematically show perspective views of one embodiment of the PBR of the invention (floating PBR) in “flat” (3A), “green-house” (3B) and “spread” (3C) configurations;

FIGS. 4A to 4C schematically show perspective views of another embodiment of the PBR of the invention (anchored PBR) in “flat” (3A), “green-house” (3B) and “spread” (3C) configurations;

FIGS. 5A and 5B schematically show a top view and a perspective view of the system of the invention for mass production of microalgal biomass; and

FIGS. 6A to 6B schematically show two perspective views of a system including four PBRs of the invention, arranged for growing and collecting the algal biomass.

### DETAILED DESCRIPTION OF THE INVENTION

The present invention provides an apparatus, a system and a method for growing microalgae. It was found by the inventors that the cost of microalgal culturing systems as well as the growth efficiency of microalgae can be significantly improved by using: i) an elongated transparent flexible photobioreactor (PBR) laying in a body of water; ii) an elongated internal gas dispensing system having several apertures and providing gas bubbles all along the horizontal fluid containing body; iii) at least one internal elongated draining/filling system, comprising either two separate tubes or one single tube, and having several apertures and used for filling or draining medium all along said PBR; and optionally iv) floating means enabling the stabilization of said PBR in said body of water according to the shape configuration of said PBR.

The body of the PBR is an elongated transparent flexible element having a sleeve shape, which is closed at both ends by any conventional method known in the art, such as welding, gluing, etc. The elongated transparent flexible element can be also welded on its length to create several small containing bodies with the same element, each of said small bodies being closed at both ends with conventional methods and forming a separate PBR. Modifications, such as welding, on the side of the elongated transparent flexible body, should be minimal, as it would decrease the PBR reliability, increase its production costs, reduce its flexibility, and restrain its ability to adopt different shapes and configurations. The bodies of the PBR of the invention have a low production cost and are preferably made of a plastic material such as polyethylene. The body of water is merely used as a temperature buffering element but may also be used to control the shape configuration of the PBR.

The elongated gas dispensing system is placed inside the PBR body, all along its length, and provides a gas mixture which is used both for providing an effective low-shear force mixing of the growth solution and furnishing essential gaseous nutrients to the algae population. The gas apertures of the gas dispensing system are preferably facing towards the bottom of the PBR body, thereby avoiding entrance of solution when the gas flow is stopped. In one embodiment of the PBR of the invention, the gas dispensing system comprises a single tube which provides large bubbles (diameter of about 2.5 cm or above) of a gas mixture comprising air, enriched with different gaseous components, e.g., carbon dioxide, nitrogen dioxide. The different gaseous components that are used to enrich the air, may be provided by commercial systems, gas emissions from industrial plants, coal power plants, or other sources. In another embodiment of the PBR of the invention, the gas dispensing system comprises two different tubes, the first one providing large gas bubbles of air to achieve a low stress mixing of the growth solution, and the second one providing microbubbles (diameter of about 0.5 cm or below) of a gas mixture comprising air, enriched with different gaseous components, e.g., carbon dioxide, nitrogen dioxide, used as nutrients by the algae. In some embodiments of the PBR of the invention, the gas dispensing system is a polyethylene tube placed in the upper part of the PBR, which comprises vertical microtubes which are dipped into the algae-containing solution. The microtubes are maintained in a vertical position either by placing microweights at the end of each microtubes or by fixing said microtubes to an element placed at the bottom of the PBR (for instance, the draining element), or to the bottom of the PBR body itself. In other embodiments, the gas dispensing system is placed at the bottom of the PBR and comprises, optionally, microweigths to avoid it floating above the algae-containing solution.

The mixing efficiency of the liquid suspension contained in the PBR by using gas bubbles depends merely on the side of said bubbles. In the present invention, the gas dispensing system produces preferably large gas bubbles with a diameter of about 2.5 cm or more. These large bubbles have a high volume/surface ratio and move up rapidly in the liquid solution containing the algae, thereby enabling efficient mixing of said solution. The diameter of the bubbles depends on the size of the apertures of the gas dispensing system and on the gas flow rate. If considering a specific gas dispensing system with apertures having a specific size, a low gas flow rate will result in the production of small bubbles, whereas a high gas flow rate will produce larger bubbles. However, maintaining a constant high gas flow rate in a large-scale system is energy consuming and costly. In order to reduce the energy consumption of the system, the present invention enables the production of large gas bubbles by pulses. In that way, efficient mixing may be achieved and the energy consumption is considerably reduced. Moreover, the frequency of said pulses can be controlled and adapted to particular conditions, such as time in the day, growth cycle stage, etc. When gas bubbles are not produced, the growth solution cannot enter into the gas dispensing system as the gas apertures are facing the bottom of the PBR body.

The elongated filling/draining system present in the PBR of the invention comprises either one single tube which is used for both filling and draining the solution contained in the PBR, or a pair of tubes, each one dedicated to a specific task, namely filling the PBR with growth medium comprising fertilizing agents, and harvesting the growth solution from the PBR. Furthermore, said elongated filling/draining system is used to control the volume of solution present in the PBR, thereby obtaining different PBR profiles/shape configurations of the PBR inside the body of water. The filling/draining element, which preferably comprises more than one aperture, enables the exchange of medium all along the transparent body of the PBR, thereby keeping homogenous conditions all along said PBR; moreover, since the movement of medium is restricted to the environment of said apertures, the risk of developing contaminants is reduced.

To the knowledge of the inventors, the present invention is the only one to provide an horizontal flexible PBR having a vertical gas mixing of the growth solution and a parallel filling/draining all along said PBR. The surface to volume ratio (s/v ratio) of the flexible PBR of the invention can be adapted to provide optimal growth conditions to different algal species or different stages of the growing cycle. Ideal growth conditions can be maintained in the PBR despite variations of the external conditions, such as temperature, light exposition, etc., by varying the volume ratio of solution/gas inside the elongated body, by varying the level of the body of water in which the PBR is laying into, and by optionally using floating means that can be inflated or deflated to stabilize said PBR in said body of water.

The PBR of the invention can be used for growing and collecting any photosynthetic microorganisms, and in particular microalgae. The concentration of the microalgal population is preferably maintained at a constant level in order to maximize the photosynthetic rate. This may be achieved by adding fresh growth medium or by draining some liquid suspension according to the density of the algal population, by using the above-described draining/filling system.

The present invention also provides several advantages regarding PBR sanitization. All growth systems are exposed to contaminants. While open pounds used for growing algae are more subject to contaminations, closed system may also develop unwanted microorganisms, which results in decreasing growth efficiency and culture purity. Therefore, all PBRs should be sanitized either preventively or when a contamination is suspected or observed. Generally, such contaminants are localized on the surface of the PBR and the cleaning material should be brought into contact with said surfaces. In open pounds, the growth solution, containing the algae, is drained out of the pound and the walls may be disinfected by applying an appropriate disinfecting material. In the case of open pounds, the volume of cleaning solution is relatively small if compared to the surfaces that should be cleaned. In most of the closed PBRs of the prior art, which are composed of non-flexible bodies, all the PBR volume should be filled with the disinfecting solution in order to clean the internal surfaces, which results in a huge consumption of cleaning material. Conversely, sanitization of the PBR of the present invention is realized by deflating and emptying the flexible PBR, and by introducing a small amount of cleaning material in the almost flat internal space. The sanitization can be done either by introducing a cleaning solution via the medium filling tube or by introducing a gaseous mixture (e.g. ozone or chlorine) through the gas dispensing system. Therefore, and in contrast with existing PBRs, only a small volume of sanitizing agent is necessary to achieve a satisfying level of sanitization of the PBR of the invention.

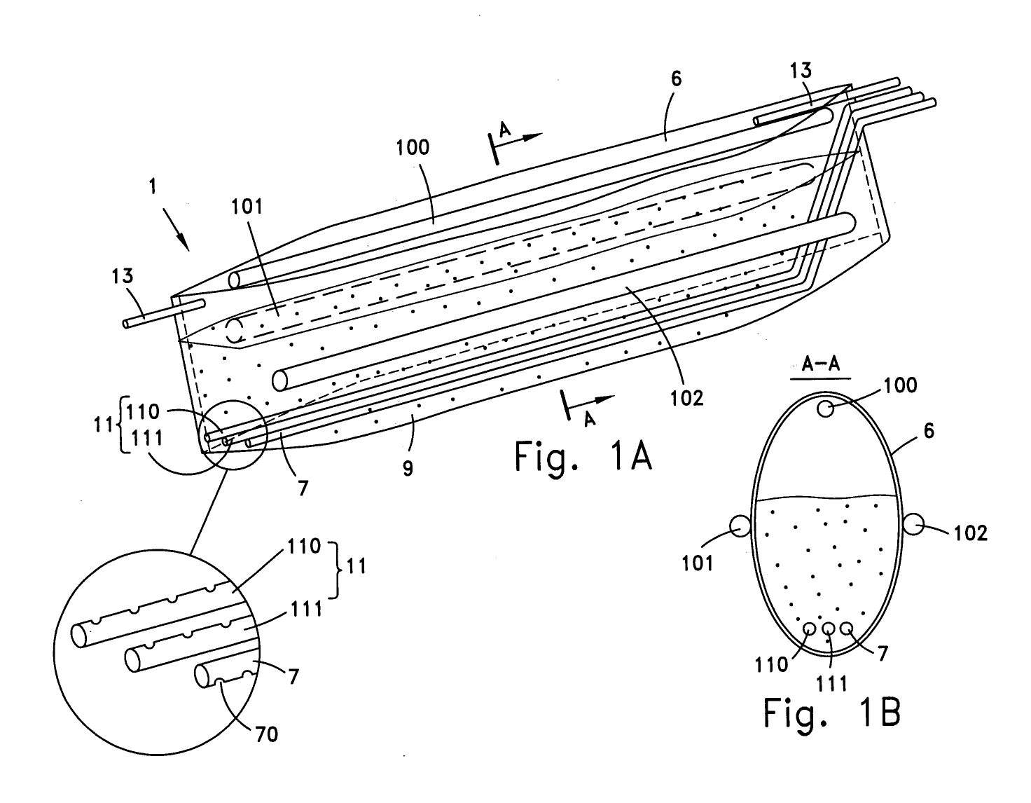

Referring to FIGS. 1A and 1B, shown is one embodiment of the PBR 1 of the invention comprising:
i) a transparent elongated body 6 made of a transparent, flexible material, used for containing the microalgae in a liquid suspension;
ii) an elongated gas dispensing system 7, placed at the bottom of said body 6, and used for conveying a gas mixture and releasing it as gas bubbles by means of at least one gas aperture 70 positioned along said gas dispensing system 7;
iii) an elongated draining/filling system 11, placed at the bottom of said body 6, and comprising a filling tube 111 and a draining tube 110, which are used to add a volume of growth medium or remove a volume of liquid suspension respectively;
iv) two gas evacuation openings 13 situated at both ends of the transparent body 6, and enabling degassing of the excess of oxygen produced during the photosynthesis; monitoring the debit rate of gas going out through gas evacuation opening 13 allows the control of the volume of gas accumulated into the transparent body 6; and
v) floating means 100, 101 and 102, which have either a permanent shape (element 100) or are connected to a system that allows their inflation or deflation according to the external conditions, by movement of an internal gas, typically air (elements 101 and 102).

In the embodiment of the PBR of the invention shown in FIGS. 1A and 1B, the transparent body 6 is made of a 400 μm thick flexible polyethylene sleeve, having a width of 40 cm, and a length of about 10 m. Said transparent body 6 can contain approximately 500 L of liquid solution but is preferably filled up to 60-70% by the growth solution. The floating means 100 situated in the upper part of the PBR, when present, is preferably formed by a 25 mm grade 4 polyethylene tube, closed at both ends. The draining/filling system 11 comprises two 16 mm PVC tubes used as draining tube 110 and filing tube 111 respectively, each tube comprising 1 mm holes preferably positioned every 4 cm. The gas dispensing system 7 is a 16 mm grade 4 polyethylene tube situated at the bottom of the transparent containing body 6, and having gas apertures being positioned preferably every 10-20 cm along said gas dispensing system 7, and is able to provide a gas flow rate of up to 20 L/h. In some embodiments of the PBR of the invention, the gas dispensing system 7 comprises additional weights to maintain the tube at the bottom of the containing body 6. As will be appreciate by a skilled person, any dimensions given herein are examples and are not intended to limit the invention in any way, being understood that the invention can be carried out using elements of any suitable dimension.

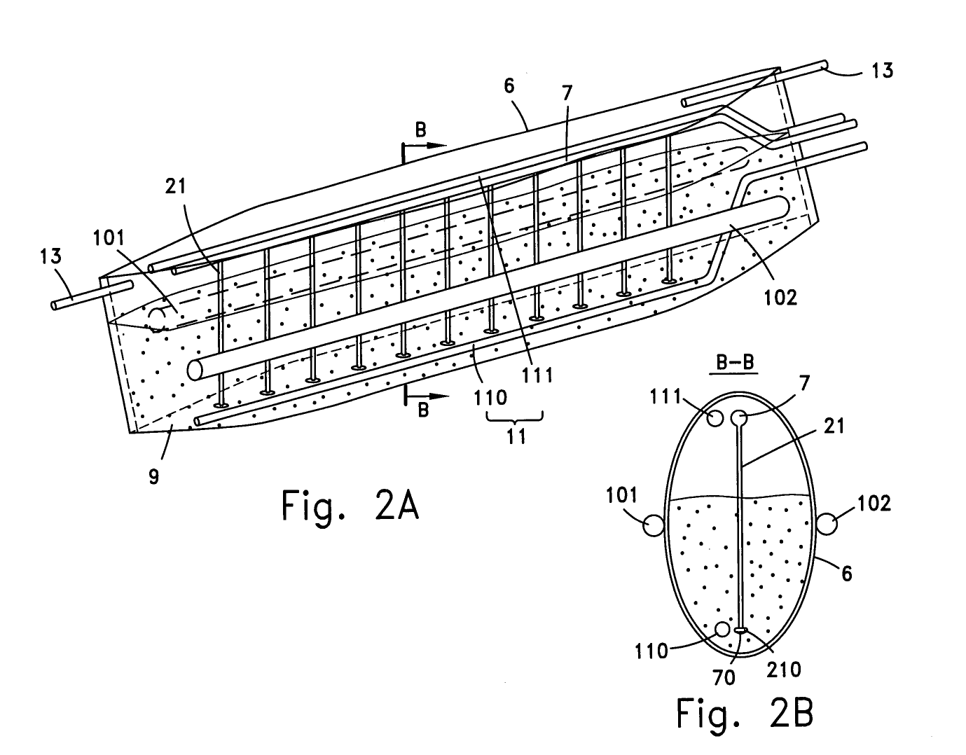

Referring to FIGS. 2A and 2B, shown is another embodiment of the PBR 1 of the invention comprising:
i) a transparent elongated body 6 made of a transparent, flexible material, used for containing the microalgae in a liquid suspension;
ii) an elongated gas dispensing system 7, placed in the upper part of said body 6, and comprising lateral microtubes 21 which are dipped into the growth solution 9, each of said microtubes having at least one gas aperture 70 and being maintained in a vertical position by a microweight 210;
iii) an elongated draining/filling system 11 comprising a draining tube 110 placed at the bottom of said body 6, and a filling tube 111 placed in the upper part of said body 6;
iv) two gas evacuation openings 13 situated at both ends of the transparent body 6; and
v) floating means 101 and 102, which are connected to a system that allows their inflation or deflation according to the external conditions, by movement of an internal gas, typically air.

In the embodiment of the PBR of the invention shown in FIGS. 2A and 2B, the gas dispensing system 7 is a 16 mm grade 4 polyethylene tube with additional vertical lateral polyethylene microtubes 21. In this embodiment, the gas dispensing system 7 may act as a floating means to stabilize the structure of the PBR according to its particular shape configuration. As will be appreciate by a skilled person, any dimensions given herein are examples and are not intended to limit the invention in any way, being understood that the invention can be carried out using elements of other suitable dimensions.

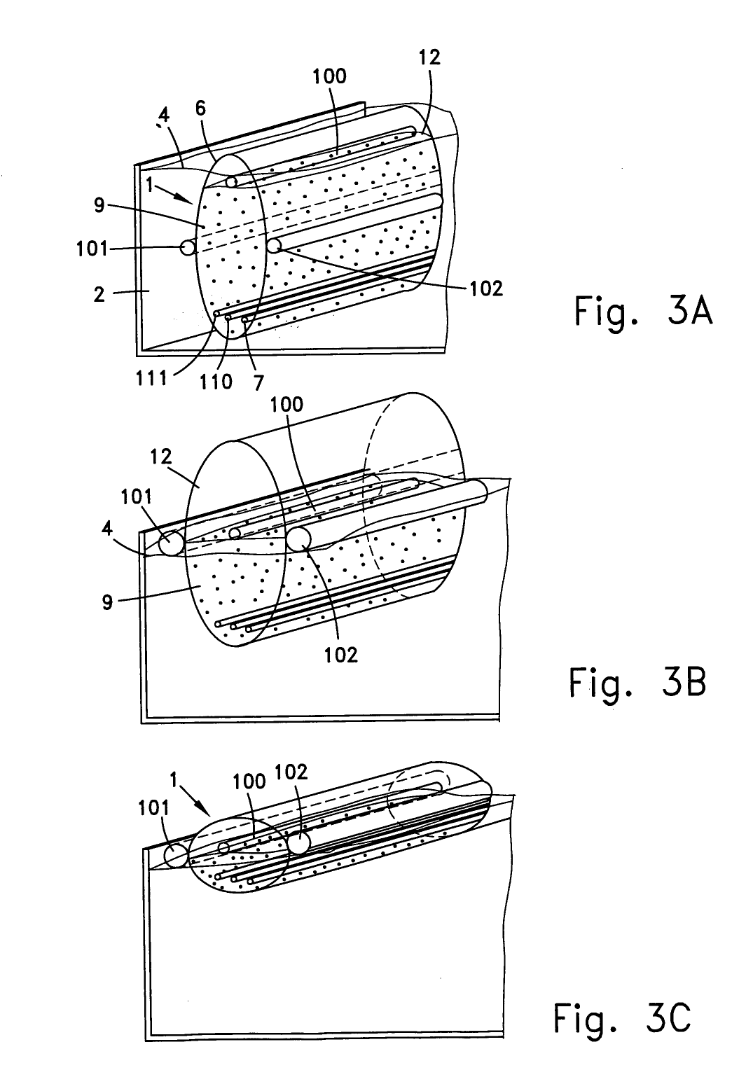

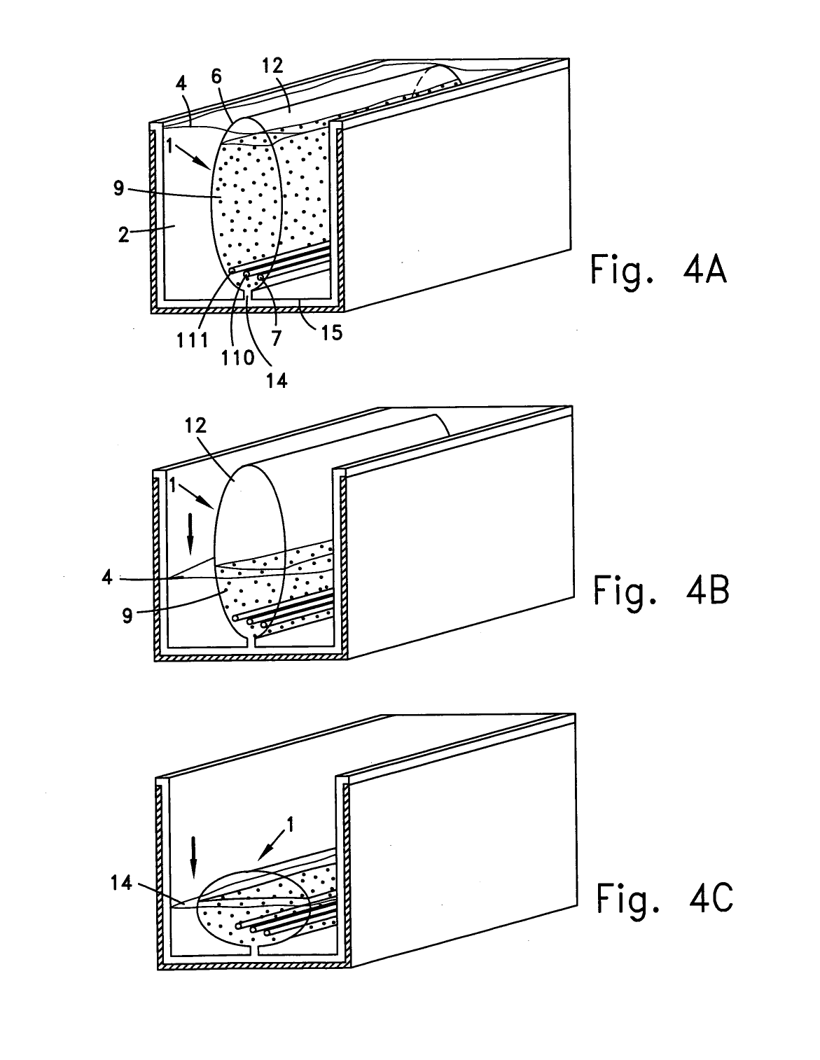

Referring to FIGS. 3A and 4A, shown are perspective views of two specific embodiments of the PBR 1 of the invention (respectively a floating PBR and an anchored PBR), in “flat” configuration. Both are composed of a transparent body 6, a draining/filling system 11, a gas dispensing system 7 and are immersed in a body of water 2, having a water level 4. A liquid phase of microalgal growth solution 9 shares the internal volume of the transparent body 6 with a gas layer 12 which is present above. The floating PBR is preferentially provided with at least an upper floating means 100 and at least two side floating means 101 and 102, which allows maintaining the same buoyancy level all along said transparent body 6. The floating PBR may be used in any kind of water body, natural or artificial, e.g. sea, lakes, water reservoirs. In contrast, the anchored PBR is preferentially used in custom-made water ponds. The anchored PBR has an anchoring means 14 that maintains it close to the bottom 15 below the body of water 2. Optionally, both the bottom 15 and the transparent body 6 of the anchored PBR are made of a single sheet of transparent flexible material.

Referring to FIGS. 3A-3C and 4A-4C, shown are perspective views of the floating PBR and the anchored PBR of the invention, in different shape configurations, which are switched according to the environmental conditions, i.e. enlightening, temperature, etc. In normal conditions (FIGS. 3A and 4A), the PBRs are immersed into the body of water 2 maintaining the culture medium at a constant temperature. In FIG. 3A, the floating means 100 allows the PBR 1 to adopt an almost flat vertical shape into said water. In 4A this shape is obtained by the combined action of the gas layer 12 at the top end of the PBR 1 and the tension exerted by the anchoring means 14. Under low temperature and weak light exposure (FIGS. 3B and 4B), a thick gas layer 12, which acts as an insulating layer, is created upon the growth solution 9, for instance by lowering the level of the gas evacuation openings, thereby accumulating gas in the upper part of the transparent body 6 (see FIG. 1A or 2A, gas opening 13). The thick gas layer 12 causes the floating PBR (FIG. 3B) to emerge of about half above the water level 4, the level of buoyancy all along the transparent body 6 being maintained by the inflation of the side floating means 101 and 102. In the case of the anchored PBR (FIG. 4B), the water level 4 is decreased to expose more PBR 1 surface to the light and the gas evacuation opening are lowered. With normal temperature but weak light exposure (FIGS. 3C and 4C), the PBR 1 adopts a spread configuration by either inflating the floating means 101 and 102 (FIG. 3C) or lowering the level of water 4 (FIG. 4C), without accumulation of gas in the upper part of the PBR 1. The person skilled in the art would clearly understand that other configurations and shapes of the PBRs of the invention may be potentially obtained, and that the above examples have been described for illustrative purpose only.

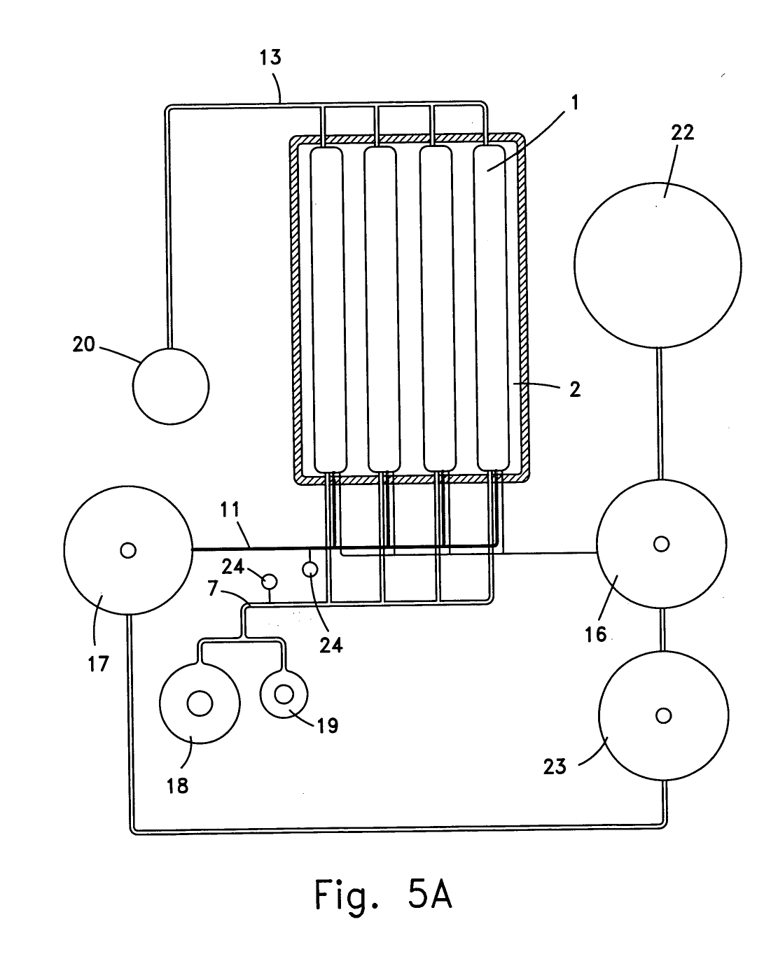

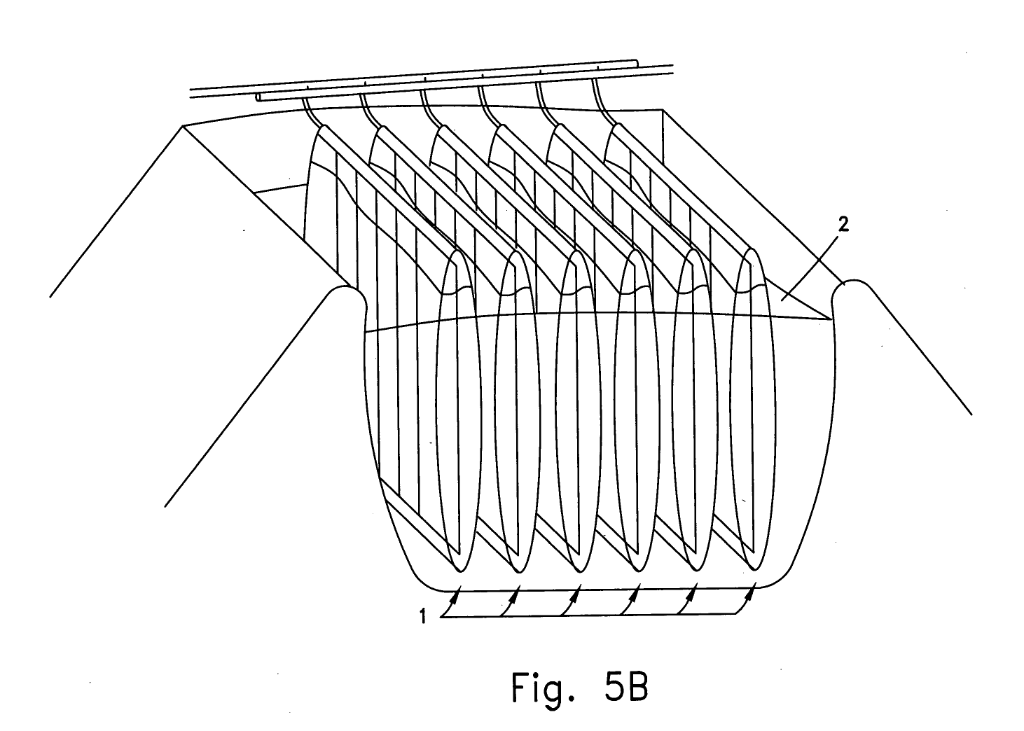

Referring to FIG. 5A, shown is a top view of one embodiment of the system of the invention, which comprises:
i) at least two PBRs 1 positioned horizontally in a body of water 2;
ii) a miscellaneous gas providing system 18 and an air providing system 19, the miscellaneous gas and the air being mixed in a specific ratio in the gas dispensing system 7; the gas mixture is brought to the PBRs 1 and is released through gas apertures in the form of bubbles, the debit of the gas mixture being adjustable;
iii) a growth medium supplying system 17 that provides fresh growth medium to the growth solution by means of the draining/filling system 11;
iv) a storage system 22, which stores the algal concentrate harvested from the PBRs 1 until its use in the factory.
v) a medium recycling system 23, which recycles the growth medium drained from the PBRs 1, and transfers it to said growth medium supplying system 17;
vi) a collecting system 16 that collects the solution drained from the PBRs 1 through the draining/filling system 11, and redirects the algal concentrate to said storage system 22 and the remaining growth medium to said medium recycling system 23; and
vii) a degassing system 20 which pumps or simply evacuates the exceeding volume of gas present in the PBRs through the gas evacuation openings 13, the debit of gas evacuation being adjustable.

The air providing system 19 preferably pumps the air from the environment through microfilters to avoid contamination. Up to 1% miscellaneous gas are injected into the gas distribution tube, this ratio being adjustable according to the required growth conditions.

Referring to FIG. 5B, shown is a perspective view of a part of the system of the invention comprising the PBRs 1 and the body of water 2.

The invention also provides a method for the cultivation of microalgae into PBRs, or systems comprising them, comprising the steps of:
i) introducing microalgae in a liquid suspension containing a growth medium into an elongated transparent PBRs laying horizontally on a body of water;
ii) exposing said bioreactor to the light (typically sunlight), allowing sufficient light to pass through said liquid suspension and enabling photosynthetic algae to perform photosynthesis;
iii) mixing said solution by means of gas bubbles, and simultaneously providing gaseous nutrients in an enriched air mixture by means of a tubular gas dispenser with several gas aperture;
iv) filling or draining culture medium homogeneously along said PBR in order to maintain the required density of algal population; and
v) changing the cross-section and the buoyancy of said PBRs in said water body according to the external conditions (for instance, light exposure, air temperature, water temperature, PBR temperature, pH, dissolved CO₂, dissolved O₂, light absorption into the reactor) in order to maintain optimal growth conditions.

The invention further provides methods for collecting the algal biomass and recycling the remaining growth solution which has been used in the PBR of the invention. These methods are of particular interest as it enables the separation of the algal biomass without collecting all the growth culture, and avoid the use of costly techniques such as centrifugation or addition of flocculants to huge volumes of collected growth culture.

One collection method, using the PBR of the invention, comprises the steps of:
i) closing the gas dispensing system as well as the gas evacuations present in the PBR, thereby stopping mixing the growth solution and elevating the concentration of dissolved oxygen, which results in flocculation of the algal population;
ii) enabling the algal flocculates to go down to the bottom of the PBR and to accumulate in the region next to the draining tube;
iii) draining out the solution with said draining tube during a determined period of time, thus collecting only an enriched solution containing a high concentration of algae and sending it to the storage system;
iv) reopening the gas dispensing system as well as the gas evacuation, thereby enabling mixing the algal-free solution and enabling degassing of said solution, especially from dissolved oxygen; and
v) introducing a concentrated algal solution into the remaining growth solution; and
vi) optionally, introducing a volume of enriched growth medium.

Another collection method, using the PBR of the invention, comprises the steps of:
i) closing the gas dispensing system as well as the gas evacuations present in the PBR, thereby stopping mixing the growth solution and elevating the concentration of dissolved oxygen, which results in flocculation of the algal population;
ii) enabling the algal flocculates to go down to the bottom of the PBR and to accumulate in the region next to the draining tube;
iii) draining out the solution with said draining tube during a determined period of time, thus collecting only an enriched solution containing a high concentration of algae, and sending it to the storage system;
iv) draining out the remaining growth medium and sending it to the medium recycling system;
v) introducing a volume of growth solution comprising a mixture of growth medium and an algal population; and
vi) reopening the gas dispensing system as well as the gas evacuation.

In specific cases, when the algae are grown up in large diameter PBRs, the above-described methods might not be efficient, as the time spent by the algal population situated at the top of the growth solution to reach the draining tube is relatively long. Therefore, the first step that may be required in those cases, is the transfer of the whole growth solution from the large PBR (growing PBR) to several smaller PBRs (harvesting PBRs), in which the above-described methods will be performed. These harvesting PBRs may be built, for instance, by taking a transparent elongated containing body generally used for the large diameter PBR, and by welding all along said body to form several smaller PBRs, which contains all the elements as above-described.

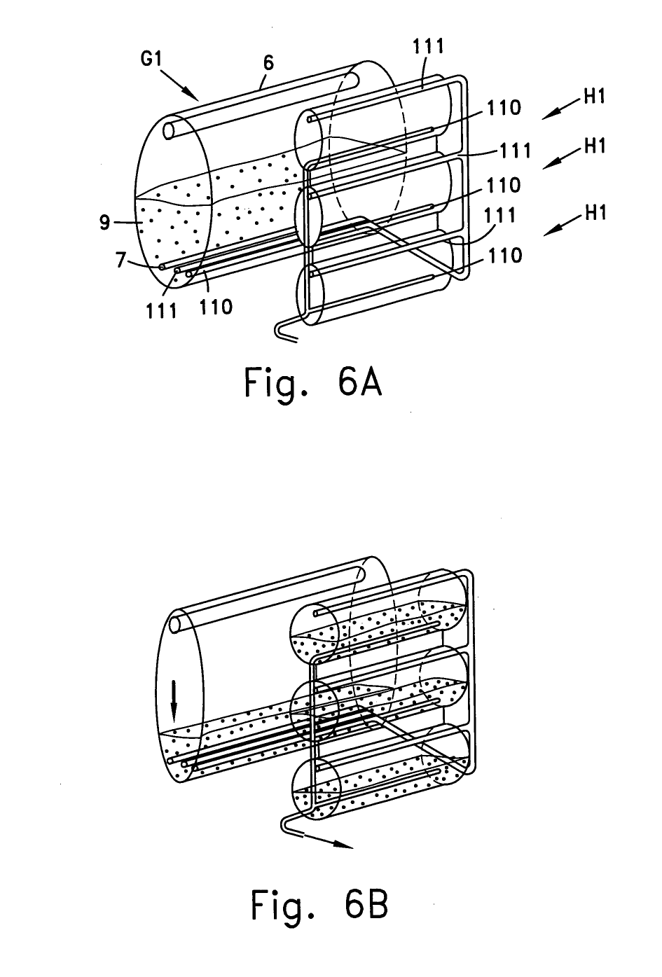

The transfer and collection of the growth solution from the growing PBR to the harvesting PBRs can be done either in a parallel mode, direct mode, or hybrid mode:
i) in parallel mode (as shown in FIGS. 6A and 6B), the growth solution 9 is drained from the growing PBR G1 and conveyed to the harvesting PBRs H1 by a system which ends into said harvesting PBRs H1 with a filling tube 111 comprising several apertures, thereby enabling filling said harvesting PBRs H1 with the drained solution at different position at the same time. The gas dispensing system 7 in said harvesting PBRs H1 is closed and the algal population is allowed to go down to the bottom of said PBRs H1 by flocculation. In the first step, the algal concentrate is collected all along said harvesting PBR by the draining tube 110 and is directed to the storage system; then, the remaining growth medium is collected by the same draining tube 110 and directed to the medium recycling system;
ii) in direct mode, the growth solution drained from the growing PBR is conveyed to the harvesting PBRs by a system which ends into the harvesting PBRs with a filling tube with one single aperture. The gas dispensing system in said harvesting PBRs is closed and the algal population is allowed to go down to the bottom of said PBRs by flocculation. As a first step, the algal concentrate is collected by a draining tube having several apertures and is directed to the storage system; then, the remaining growth medium is collected by the same draining tube and directed to the medium recycling system;
iii) in hybrid mode, the growth solution drained from the growing PBR is conveyed to the harvesting PBRs by a system which ends into the harvesting PBRs with a filling tube with one single aperture. The gas dispensing system in said harvesting PBRs is closed and the algal population is allowed to go down to the bottom of said PBRs by flocculation. As a first step, the algal concentrate is collected all by a draining tube having several apertures and is directed to the storage system; then, the remaining growth medium is collected by another draining tube having one single aperture and directed to the medium recycling system;

While the invention has been described using some specific examples, many modifications and variations are possible. It is therefore understood that the invention is not intended to be limited in any way, other than by the scope of the appended claims.

1. An horizontal sleeve photobioreactor for growing photosynthetic microorganisms, said photobioreactor comprising:
i) a flexible transparent elongated body suitable to contain a liquid suspension of photosynthetic microorganisms, and adapted to be positioned horizontally in a body of water;
ii) an elongated gas dispensing system for providing nutrients by means of bubbling a gas mixture to a liquid suspension and for performing a vertical low stress mixing of said liquid suspension all along said elongated body;
iii) an elongated filling/draining system for providing a growth medium and collecting said liquid suspension all along said elongated body; and
iv) at least one gas evacuation opening;
characterized in that both the cross-section and buoyancy of said flexible elongated body is suitable to be modified in real-time by adjusting the volumes of gas and liquid contained therein.

2. A photobioreactor according to claim 1, wherein said elongated gas dispensing system comprises a single gas dispensing tube which is placed at the bottom of said photobioreactor.

3. A photobioreactor according to claim 2, wherein said gas tube comprises one or more weights.

4. A photobioreactor according to claim 2, wherein said gas dispensing tube comprises one or more gas apertures facing the bottom of said photobioreactor.

5. A photobioreactor according to claim 1, wherein said gas dispensing system comprises a first and a second gas dispensing tube, both of said tubes being placed at the bottom of said photobioreactor and comprising one or more gas apertures facing the bottom of said photobioreactor.

6. A photobioreactor according to claim 5, wherein said first gas dispensing tube is suitable to provide large bubbles for achieving a low-stress mixing of said liquid suspension, and said second gas dispensing tube is suitable to provide microbubbles containing nutrients to said liquid suspension.

7. A photobioreactor according to claim 1, wherein said elongated gas dispensing system is placed in the upper part of said photobioreactor and comprises lateral microtubes.

8. A photobioreactor according to claim 7, wherein said lateral microtubes incorporate one or more weights.

9. A photobioreactor according to claim 7, wherein said lateral microtubes are fixed onto the draining tube of said filling/draining system.

10. A photobioreactor according to claim 7, wherein said lateral microtubes are fixed to the bottom of said photobioreactor.

11. A photobioreactor according to claim 1, wherein said filling/draining system comprises a single tube with one or more apertures to be used for both filling and draining a solution from said photobioreactor.

12. A photobioreactor according to claim 1, wherein said filling/draining system comprises a draining tube and a filling tube with one or more apertures.

13. A photobioreactor according to claim 12, wherein said draining tube and said filling tube are both placed at the bottom of said photobioreactor.

14. A photobioreactor according to claim 12, wherein said draining tube is placed at the bottom of said photobioreactor and said filling tube is placed in the upper part of said photobioreactor.

15. A photobioreactor according to claim 1, wherein said gas mixture can be accumulated above the liquid suspension, thereby enabling changes in the shape configuration of said photobioreactor and changes of the buoyancy of said photobioreactor in said body of water.

16. A photobioreactor according to claim 1, comprising the following elements:
i) an elongated containing body made of a transparent sleeve of a flexible material, containing photosynthetic microorganisms in a liquid suspension;
ii) an elongated gas dispensing system comprising at least one gas dispensing tube with one or more gas apertures;
iii) an elongated draining/filling system comprising at least one tube with one or more apertures; and
iv) at least one gas evacuation opening;
wherein said elongated containing body is closed at both ends and lays horizontally in a body of water, its cross-section, buoyancy, and surface-to-volume ratio, being adapted according to the external conditions and the growth cycle of said microorganisms.

17. A photobioreactor according to claim 16, further comprising at least one floating means, which may be inflated or deflated to maintain the same level of buoyancy all along said transparent containing body in said body of water.

18. A photobioreactor according to claim 16, wherein said elongated containing body is made of a weldable material.

19. A photobioreactor according to claim 18, wherein the weldable material is polyethylene.

20. A photobioreactor according to claim 16, further comprising an anchoring means which connect said photobioreactor to the bottom of an artificial water pond.

21. A photobioreactor according to claim 20, wherein said anchoring means, said water ponds and said transparent elongated body of said photobioreactor are made of a single transparent flexible element.

22. A system for the large-scale production of microalgal biomass comprising:
i) a plurality of photobioreactors according to claim 1;
ii) a miscellaneous gas providing system;
iii) an air providing system;
iv) a growth medium supplying system;
v) a storage system;
vi) a medium recycling system;
vii) a collecting system;
viii) a sanitizing system; and
ix) optionally, a degassing system;
wherein said photobioreactors lay horizontally in a body of water, and wherein all the elements of said system are connected by fluid conveying tubes.

23. A method for the cultivation of a microalgal biomass, comprising growing microalgae in a photobioreactor according to claim 1, wherein said photobioreactor is laying horizontally in a body of water, and adapting the amount of light delivered to said biomass as well as the growth temperature of said biomass by changing the cross-section, buoyancy, and/or surface-to-volume ratio of said photobioreactor in said body of water.

24. A method according to claim 23, comprising the steps of:
i) introducing a liquid suspension of microalgae containing a growth medium into an elongated transparent photobioreactor laying horizontally in a body of water;
ii) exposing said bioreactor to light, allowing sufficient light to pass through said liquid suspension and enabling photosynthetic algae to perform photosynthesis;
iii) blowing into said solution a gas mixture containing carbon dioxide by means of a gas dispensing system with several gas apertures, providing nutrients for the photosynthetic process and mixing said solution by means of bubbles;
iv) filling or draining culture medium homogeneously along said photobioreactor in order to maintain a constant density of algal population; and
v) changing the cross-section, buoyancy, and/or surface-to-volume ratio of said photobioreactor in said water body according to the external conditions;
wherein changes in the cross-section, buoyancy, and/or surface-to-volume ratio of said photobioreactor allow to monitor the amount of light delivered to the algal biomass and the temperature of the liquid suspension.

25. A method according to claim 24, wherein said gas mixture is provided in pulse mode.

26. A method according to claim 24, wherein said changes in cross-section, buoyancy, and/or surface-to-volume ratio of said photobioreactor includes modifying the volume of gas contained above the liquid suspension.

27. A method according to claims 24, wherein stabilizing the cross-section, buoyancy, and/or surface-to-volume ratio of said photobioreactor includes modifying the volume of gas in floating means.

28. A method according to claims 24, wherein said changes in cross-section, buoyancy, and/or surface-to-volume ratio of the photobioreactor includes modifying the level of water.

29. A method according to claims 24, wherein said changes in cross-section, buoyancy, and/or surface-to-volume ratio of the photobioreactor includes changing the volume of growth solution.

30. A method for collecting the algal biomass from a photobioreactor according to claim 1, comprising the steps of:
i) closing the gas dispensing system as well as the gas evacuation openings;
ii) enabling the microalgae to flocculate and fall down to the bottom of said photobioreactor;
iii) draining out the solution containing said algal biomass around the draining tube;
iv) reopening the gas dispensing system as well as the gas evacuation openings; and
v) introducing a fresh microalgal suspension in the remaining growth solution; and
vi) optionally, introducing a volume of enriched growth medium into said photobioreactor.

31. A method for collecting the algal biomass from a photobioreactor according to claim 1, comprising the steps of:
i) closing the gas dispensing system as well as the gas evacuation openings;
ii) enabling the microalgae to flocculate and fall down to the bottom of said photobioreactor;
iii) draining out the solution containing said algal biomass around the draining tube;
iv) draining out the remaining growth solution;
v) introducing a fresh microalgal suspension mixed to a fresh growth solution; and
vi) reopening the gas dispensing system as well as the gas evacuation openings.

32. A method for collecting the algal biomass from a photobioreactor according to claim 1, comprising the steps of:
i) draining the whole growth solution from a first photobioreactor and dividing said solution in several volumes, each of said volume being used for filling several photobioreactors with an internal volume smaller than said first photobioreactor;
ii) collecting the algal biomass that flocculates near the draining tube in each of said smaller photobioreactors;
iii) collecting the remaining growth medium in each of said smaller photobioreactors; and
iv) introducing a fresh microalgal suspension mixed to a fresh growth solution into said first photobioreactor.

33. A method according to claim 32, wherein said filing and collecting is made according to a mode selected from the group consisting of parallel mode, direct mode and hybrid mode.
